(19) United States
(12) Patent Application Publication
(10) Pub. No.: US 2025/0041576 A1
(43) Pub. Date: Feb. 6, 2025

(54) TATTOO MACHINE COVER DEVICE

(71) Applicant: David Hoffer, Fountain Valley, CA (US)

(72) Inventor: David Hoffer, Fountain Valley, CA (US)

(21) Appl. No.: 18/229,415

(22) Filed: Aug. 2, 2023

(51) Int. Cl. A61M 37/00

(52) U.S. Cl. CPC A61M 37/0076; A61M 2209/06

(57) ABSTRACT

A tattoo machine cover device including a bag that can receive a tattoo machine. The bag has a bottom end opposite an open-mouth end. The open-mouth end provides access to an interior pocket of the bag where the tattoo machine can be placed. The bottom end has a hole which also provides access to the interior pocket and which is configured to align with a needle cartridge aperture of the tattoo machine. The bag is formed of a heat shrinkable film material that conforms to a shape of the tattoo machine when the bag is heated by a heat gun.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to protective coverings and more particularly pertains to a new protective covering for covering a tattoo machine.

### (2) Description of Related Art Including Information Disclosed Under 37 CFR 1.97 and 1.98

The prior art relates to protective coverings and film wraps. Some examples in the prior art disclose heat shrinkable films and sleeves for covering irregularly shaped articles such as hockey sticks and orthopedic casts. Some prior art also discloses plastic or elastic casings or coatings for tattoo machines intended to increase the stability of the tattoo machine in the user's hand. However, the prior art lacks a disclosure of a sterile, disposable, heat shrinkable wrap for covering a tattoo machine to increase sanitation.

### BRIEF SUMMARY OF THE INVENTION

An embodiment of the disclosure meets the needs presented above by generally comprising a bag that can receive a tattoo machine. The bag has a bottom end opposite an open-mouth end. The open-mouth end provides access to an interior pocket of the bag where the tattoo machine can be placed. The bottom end has a hole which also provides access to the interior pocket and which is configured to align with a needle cartridge aperture of the tattoo machine. The bag is formed of a heat shrinkable film material that conforms to a shape of the tattoo machine when the bag is heated by a heat gun.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of a tattoo machine cover device according to an embodiment of the disclosure.

FIG. 2 is an isometric view of an embodiment of the disclosure.

FIG. 3 is an in-use view of an embodiment of the disclosure.

FIG. 4 is an in-use view of an embodiment of the disclosure.

FIG. 5 is an in-use view of an embodiment of the disclosure.

FIG. 6 is a top view of an embodiment of the disclosure.

### DETAILED DESCRIPTION OF THE INVENTION

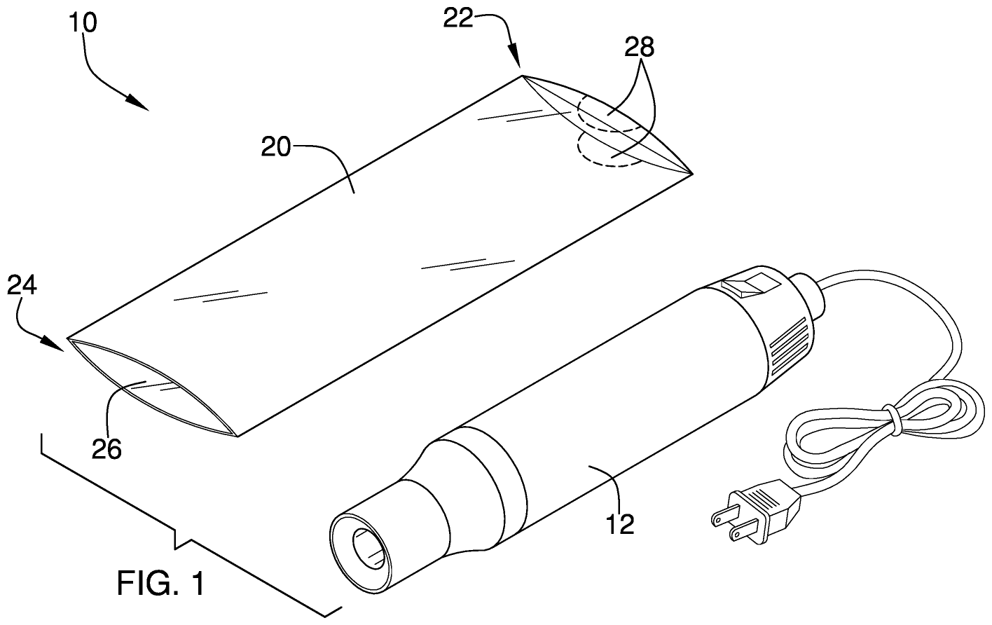

With reference now to the drawings, and in particular to FIGS. 1 through 6 thereof, a new protective covering embodying the principles and concepts of an embodiment of the disclosure and generally designated by the reference numeral 10 will be described.

As best illustrated in FIGS. 1 through 6, the tattoo machine cover device 10 generally comprises a heat gun 12, a tattoo machine 40, and a bag 20. The heat gun 12 is generally conventional and configured to emit a heated airflow.

The bag 20 has a bottom end 22 opposite an open-mouth end 24. The open-mouth end 24 provides access to an interior pocket 26 of the bag 20 where the tattoo machine 40 can be placed. The bottom end 22 has a hole 28 which also provides access to the interior pocket 26 and which is configured to align with a needle cartridge aperture 48 of the tattoo machine 40. In some embodiments, the hole 28 is also configured to receive a needle cartridge 50. The bag 20 is formed of a heat shrinkable film material, such as 100 gauge shrink wrap film, that conforms to a shape of the tattoo machine 40 when the bag 20 is heated by the heat gun 12.

The tattoo machine 40 is also generally conventional. In preferred embodiments, the tattoo machine 40 has a top side 42 opposite a bottom side 44. The top side 42 may include a connector 46 that is configured to connect with a power source. For example, the connector 46 may be a power cord or a battery port. The bottom side 44 has a needle cartridge aperture 48 therein that is configured to receive the needle cartridge 50. However, the bag 20 may be used with any tattoo machine 40 which fits within the interior pocket 26.

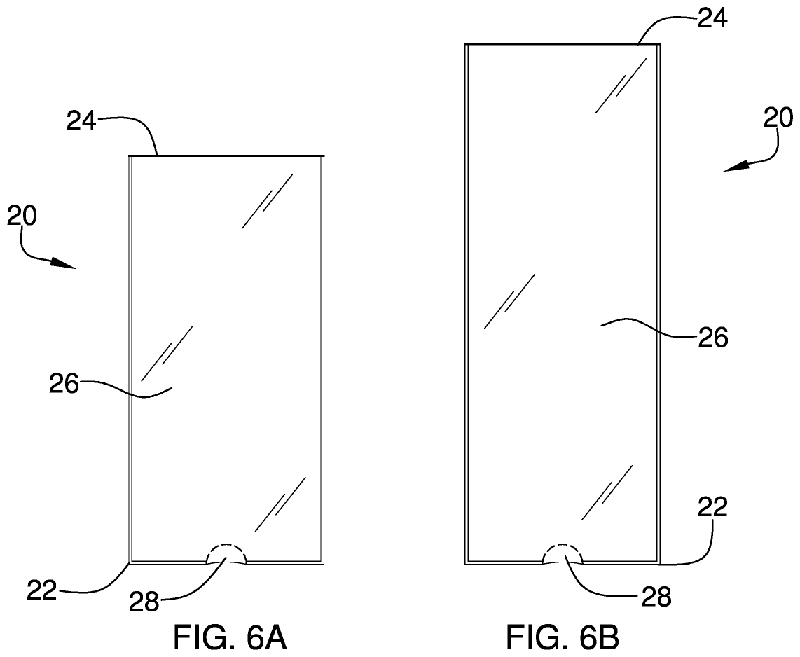

Different embodiments may include a bag 20 having different measurements and dimensions. For example, the hole 28 in the bottom end 22 of the bag 20 may have a diameter ranging from ¼ inch to ¾ inch. In one embodiment, the hole 28 has a diameter of ½ inch. Additionally, some embodiments may include a bag 20 with a generally rectangular shape having a bag width and a bag length. For example, the bag width may range from 2 inches to 4 inches and the bag length may range from 5 inches to 10 inches. In one embodiment, the bag 20 may have a bag width of 3 inches. Two example embodiments are depicted in FIG. 6. The bag 20 has a shorter bag length than the bag 20 in FIG. 6b. For example, the bag 20 in FIG. 6a may have a bag length of 6½ inches and the bag 20 in FIG. 6b may have a bag length of 8 inches. In these exemplary embodiments, the open mouth end 24 and the bottom end 22 are disposed along the bag width.

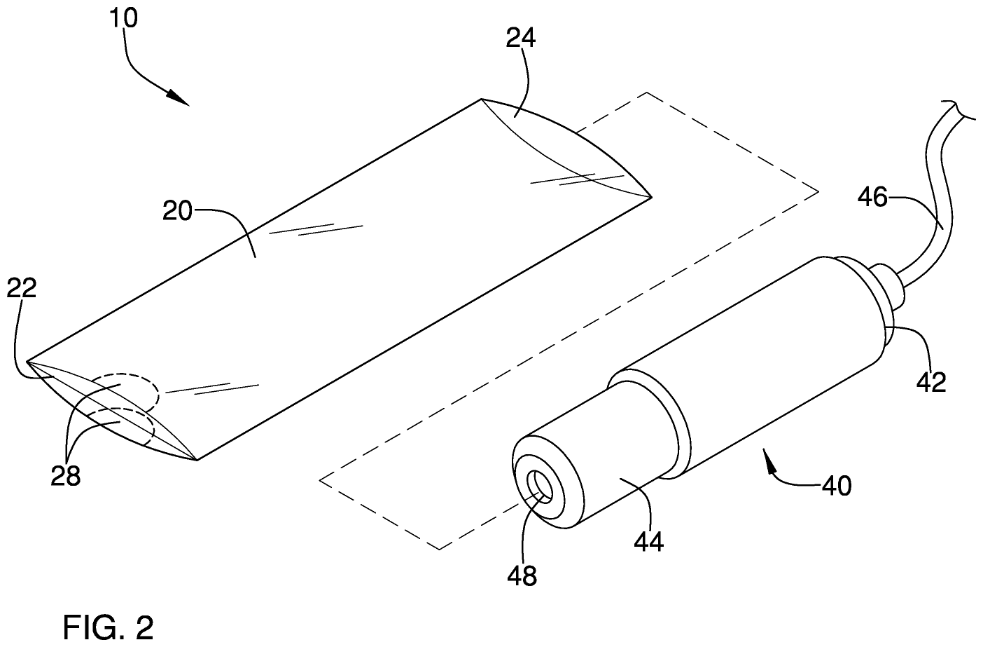

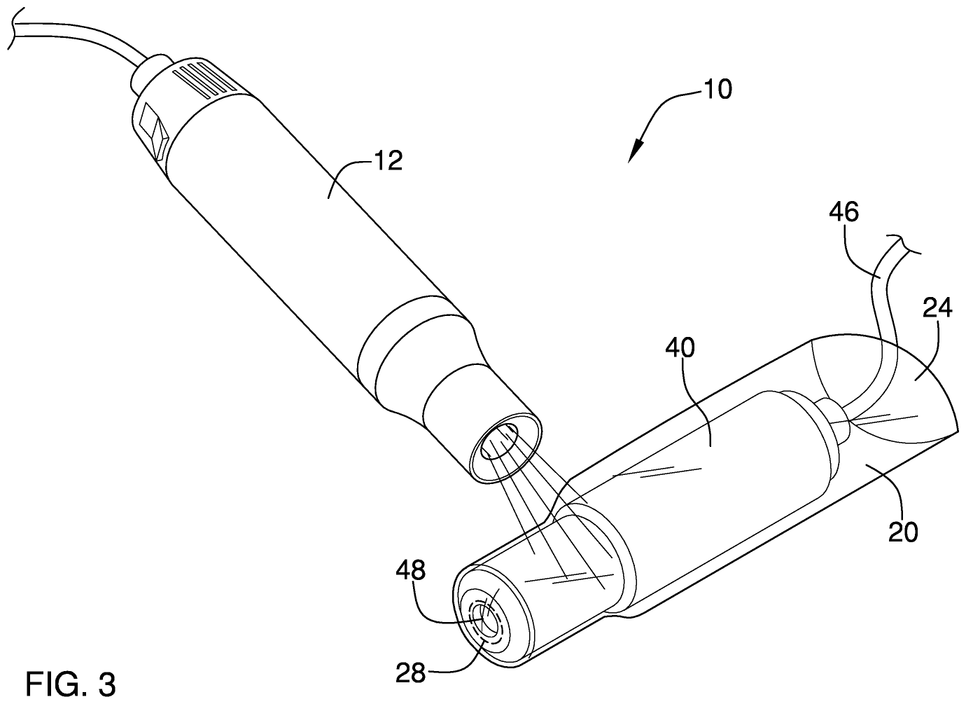

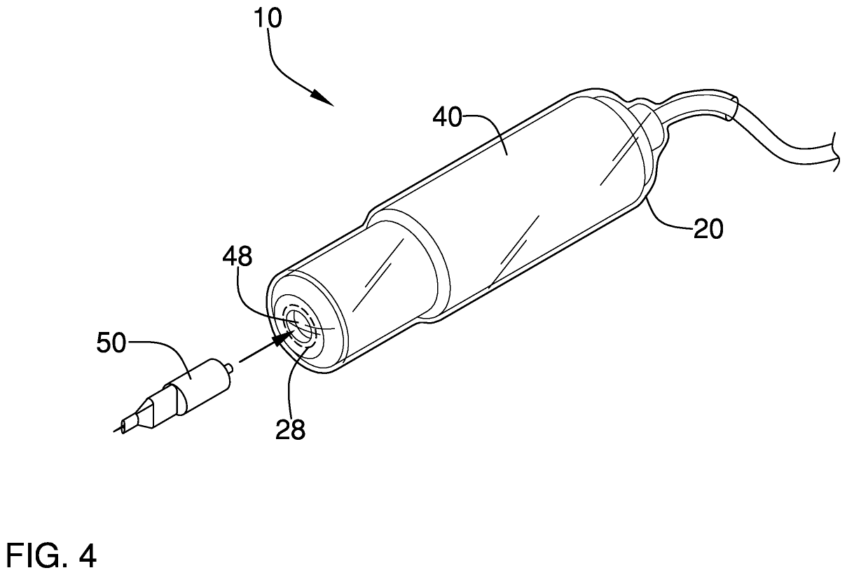

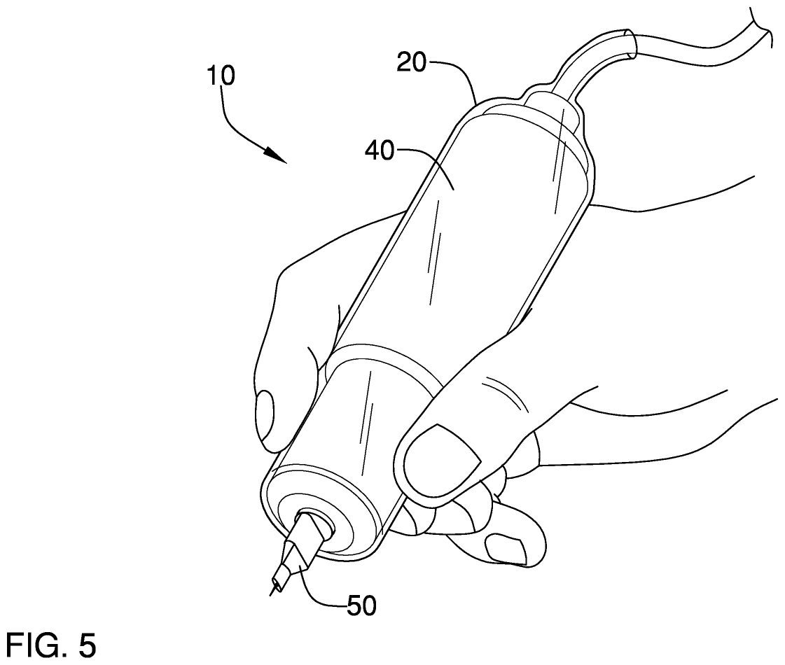

FIGS. 2-5 provide an example of the present disclosure in use. In use, the tattoo machine 40 is inserted into the bag 20 through the open-mouth end 24. For example, the bottom side 44 of the tattoo machine 40 may be inserted toward the bottom end 22 of the bag 20, such that the aperture 48 of the tattoo machine aligns with the hole 28 of the bag 20. In this configuration, the top side 42 of the tattoo machine 40 aligns with the open-mouth end 24 of the bag, such that the connector 46 of the tattoo machine 40 extends outwardly and away from the bag 20. As shown in FIG. 3, once the tattoo machine 40 is inserted into the bag 20, the heat gun 12 may be used to apply heat to the heat shrinkable film material of the bag 20. The bag 20 will then shrink and conform around the shape of the tattoo machine 40. As shown in FIG. 4, once the bag 20 has conformed to the tattoo machine 40, the needle cartridge 50 may be inserted into the aperture 48 through the hole 28 in the bottom end 22 of the bag 20. FIG. 5 depicts a hand of a user holding the fully assembled tattoo machine 40 that is covered with the bag 20. Covering the tattoo machine 40 with the bag 20 in this manner increases the cleanliness of the tattoo machine 40, reducing the risk that dirt or germs will be transferred through use of the tattoo machine 40.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A tattoo machine covering system comprising:
a heat gun;
a tattoo machine having a top side and a bottom side, the top side including a connector configured to connect with a power source, the bottom side having an aperture therein configured to receive a needle cartridge;
a bag being configured to receive the tattoo machine such that the tattoo machine is covered by the bag, the bag having a bottom end opposite an open-mouth end, the open-mouth end providing access to an interior pocket of the bag, the bottom end having a hole therein providing access to the interior pocket, the hole being configured to align with the needle aperture when the tattoo machine is inserted into the bag; and
the bag being formed of a heat shrinkable film material, the heat shrinkable film material being configured to conform to a shape of the tattoo machine when heated by the heat gun.

2. The tattoo machine covering system of claim 1, wherein the hole is further configured to receive the needle cartridge.

3. The tattoo machine covering system of claim 1, wherein the hole has a diameter ranging from ¼ inch to ¾ inch.

4. The tattoo machine covering system of claim 3, wherein the hole has a diameter of ½ inch.

5. The tattoo machine covering system of claim 1, wherein the bag has a rectangular shape including a bag width ranging from 2 inches to 4 inches and a bag length ranging from 5 inches to 10 inches.

6. The tattoo machine covering system of claim 5, wherein the bag has a bag width of 3 inches.

7. The tattoo machine covering system of claim 5, wherein the bag has a bag length of 6½ inches.

8. The tattoo machine covering system of claim 5, wherein the bag has a bag length of 8 inches.

9. The tattoo machine covering system of claim 5, wherein the open mouth end and the bottom end are disposed along the bag width,

10. The tattoo machine covering system of claim 1, wherein the heat shrinkable file material comprises 100 gauge shrink wrap film.

11. A tattoo machine covering system comprising:
a heat gun configured to emit a heated airflow;
the tattoo machine having a top side opposite a bottom side, the top side including a power cord, the bottom side having a needle aperture therein configured to receive a needle cartridge; and
a bag being configured to receive the tattoo machine such that the tattoo machine is covered by the bag with the power cord extending outwardly from the bag, the bag having a bottom end opposite an open-mouth end, the open-mouth end providing access to an interior pocket of the bag, the bottom end having a hole therein providing access to the interior pocket, the hole being configured to align with the needle aperture when the tattoo machine is inserted into the bag, the hole being further configured to receive the needle cartridge, the hole having a ½ inch diameter, wherein the bag has a rectangular shape including a bag width ranging from 2 inches to 4 inches and a bag length ranging from 5 inches to 10 inches, wherein the open mouth end and the bottom end are disposed along the width, wherein bag is formed of a heat shrinkable film material, the heat shrinkable film material being configured to conform to a shape of the tattoo machine when exposed to the heated airflow, the heat shrinkable film material comprising 100 gauge shrink wrap.

12. A method for covering a tattoo machine, the method comprising:
inserting the tattoo machine into a heat-shrink sleeve, the tattoo machine having a top side and a bottom side, the top side including a power cord, the bottom side having a needle aperture therein configured to receive a needle cartridge, the heat-shrink sleeve comprising:
a bag being configured to receive the tattoo machine such that the tattoo machine is covered by the bag, the bag having a bottom end opposite an open-mouth end, the open-mouth end providing access to an interior pocket of the bag, the bottom end having a hole therein providing access to the interior pocket, the hole being configured to align with the needle aperture when the tattoo machine is inserted into the bag; and
applying heat to the bag whereby the bag shrinks to conform to a shape of the tattoo gun.

13. The method of claim 12, further comprising inserting the needle cartridge into the needle aperture through the hole in the bottom end of the bag.
